(19) United States
(12) Patent Application Publication
(10) Pub. No.: US 2026/0094700 A1
(43) Pub. Date: Apr. 2, 2026

(54) NURSING CARE INFORMATION SYSTEM

(71) Applicant: BUDDHIST TZU CHI MEDICAL FOUNDATION, HUALIEN COUNTY (TW)

(72) Inventors: CHING HSIANG CHEN, CHIAYI COUNTY (TW); CHIH-WEI HUANG, CHIAYI COUNTY (TW); CHING-CHUNG TSAI, CHIAYI COUNTY (TW); TIN-KWANG LIN, CHIAYI COUNTY (TW); CHIA-JUNG CHEN, CHIAYI COUNTY (TW)

(21) Appl. No.: 19/314,857

(22) Filed: Aug. 29, 2025

Related U.S. Application Data
(60) Provisional application No. 63/699,887, filed on Sep. 27, 2024.

(51) Int. Cl. G16H 40/20; G16H 15/00; G16H 20/10; G16H 40/67

(52) U.S. Cl. CPC G16H 40/20; G16H 15/00; G16H 20/10; G16H 40/67

(57) ABSTRACT

A nursing care information system including mobile devices and hospital information computer devices; the mobile device includes: a user interface module electrically connected to the mobile device, for inputting user information; and six modules electrically connected to the user interface module, including a patient list module, for providing a list of patients cared for by nursing staff; a medical order confirmation module, for pushing and sound notifications, reminders and confirmations to nursing staff; a mobile drug delivery module to providing medical advice to patients under the care of nursing staff; an nursing record module to provide nursing staff with input of patient event records; a work list module to provide and remind nursing staff of to-do items; the upload photo module is used to provide nursing staff with the ability to take photos of patient wounds and image records of special events.

### FIELD OF THE INVENTION

The present invention relates to a nursing care information system, combining the characteristics of informatization, automation, visualization, and real-time, with the nursing staff as the center, greatly reducing the original problem of limited working space, but also providing timely and complete care to patients, effectively improving work efficiency and quality of care.

### BACKGROUND OF THE INVENTION

Computer dependence is high, because nursing operations have generally introduced informatization, and most tasks need to be completed by desktop computers or mobile work cart computers, including checking electronic medical records, confirming medical orders, and executing nursing records; however, these computer equipment The setting location is fixed and frequently used, causing caregivers to wait or search for available computers, affecting work efficiency.

The efficiency of medical order confirmation is low. After the doctor issues the medical order, the nursing staff needs to find a desktop computer to confirm the medical order to indicate receipt of this message and leave a time track in the computer; but usually the nursing staff must open the computer to Being able to know whether there is a new medical order still depends on the status of the computer being used at the time, and the nursing work at that time is often delayed because the computers are being used by others; if the medical order is urgent, the doctor prescribes After the procedure is completed, it is also necessary to take the time to find the patient's primary nursing staff to inform them of the new medical instructions to remind them to be implemented as soon as possible.

Work list management is cumbersome. Since the work of nursing staff is very complex and there is a lack of a work list information system that meets personal needs, nursing staff need to use paper and board clips to remind themselves of their work matters when they go to work to avoid omissions. Although paper and board clips serve as reminders, they still need to be manually sorted and copied by nursing staff, which not only increases the workload, but also easily causes omissions or errors.

Medication operations are complex and limited. The mobile work vehicle is a vehicle body equipped with a computer. The vehicle is loaded with the day's medicines and tools required for drug administration for all patients under the care of the primary nursing staff. When administering drugs, nursing staff need to push the mobile work cart to the patient's bedside and use computers and barcode scanners to perform barcode medication administration operations. The barcode is an information control function set up for patient safety considerations and can intercept wrong medicines, wrong medication time and errors. However, due to the limitations of the vehicle body, the bed space of some multi-bed beds is small, and the mobile work vehicle cannot be completely pushed to the patient's bedside. As a result, the error check information fed back by the computer cannot be received in real time, resulting in medication errors. The risk of It is physically demanding, and at night, the moving sound of the mobile work vehicle will disturb the patient's sleep; this method of drug administration not only increases the workload of nursing staff, but also reduces work efficiency, and may affect the patient's therapeutic effect and safety. Sex has a negative impact.

Nursing records are not timely enough. Nursing records are records of nursing assessments, nursing measures, and evaluation processes performed by nursing staff on patients they care for. They have the characteristics of accuracy, continuity, immediacy, clarity, and confidentiality; this means that nursing staff must truly and accurately reflect the patient's condition and nursing measures, record all nursing processes from admission to discharge in a consistent manner, and record in real time to ensure that other medical staff can understand the patient's latest status in a timely manner. The organization is clear and the language is concise and easy to understand to ensure that readers will not misunderstand it. Since the record contains the patient's personal privacy information, it should be kept strictly confidential and restricted to relevant medical staff. In addition to being a legal document, the nursing record can also be used in medical care. In addition to being used as evidence in disputes, it also provides the functions of team communication, quality management, and nursing education. According to the above characteristics, nursing records are very important and the ability to write nursing records requires professional training, but in busy nursing work Under the current situation, based on patient safety considerations, nursing staff will give priority to patient issues, so that it is difficult for most nursing staff to meet the requirements of real-time records, and they are even too busy to have time to write nursing records after work. This recording method is not only and not timely enough, but also re-collective writing is also prone to errors or omissions.

The process of taking photos and recording is cumbersome. Due to the lack of a photo uploading system, when nursing staff take care of a patient's wound, in order to record the changes before and after the wound is taken care of, they must take photos with an official camera at any time. After taking the photos, they are then connected to the computer at the nursing station with a transmission line. Finally, the photos are stored in the location specified by the system. The process is cumbersome and time-consuming, which reduces work efficiency.

The present invention provides a comprehensive, convenient and intelligent solution to the above problems.

### SUMMARY OF THE INVENTION

The present invention relates to a nursing care information system, including a mobile device and a remote computer device; the mobile device includes: a user interface module electrically connected to the mobile device, for inputting user information; and six modules electrically connected to the user interface module, including a user interface module for inputting user information, starting from the patient list module, a doctor's order confirmation module, and mobile medication Module, event recording module, work list module, and photo upload module provide nursing staff with the ability to care for patients accurately and efficiently, reduce working hours, and reduce the workload and pressure of nursing staff.

Details of the embodiments of the invention are set forth in the following drawings and descriptions to make other features, purposes and advantages of the invention more concise and easy to understand.

### BRIEF DESCRIPTION OF THE DRAWINGS

FIG. 1 shows the system architecture diagram of manufacturing steps of the present invention;

FIG. 2 shows the input interface in the mobile device;

FIG. 3 shows the screen of the patient list module;

FIG. 4 shows the screen of the medical order confirmation;

FIG. 5 shows the screen of the mobile drug delivery module;

FIG. 6 shows the screen of the work list module;

FIG. 7 shows the screen of the nursing record module; and

FIG. 8 shows the screen of the photo upload module.

### DETAILED DESCRIPTION OF THE PREFERRED EMBODIMENT

The following embodiments are used to illustrate the implementation of the present invention, and those familiar with this technology can easily understand other advantages and effects of the present invention from the content disclosed in this specification, and can also use other different specific embodiments. carry out or apply.

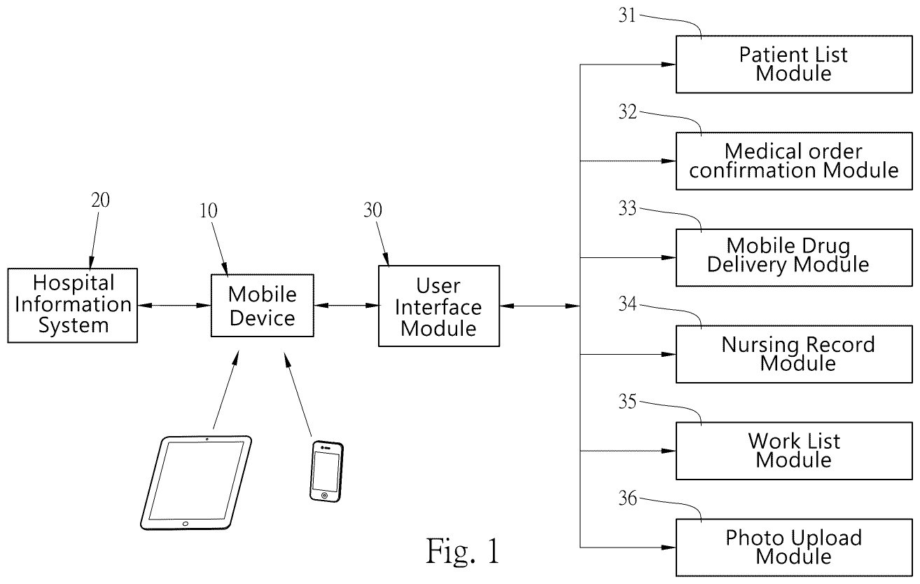

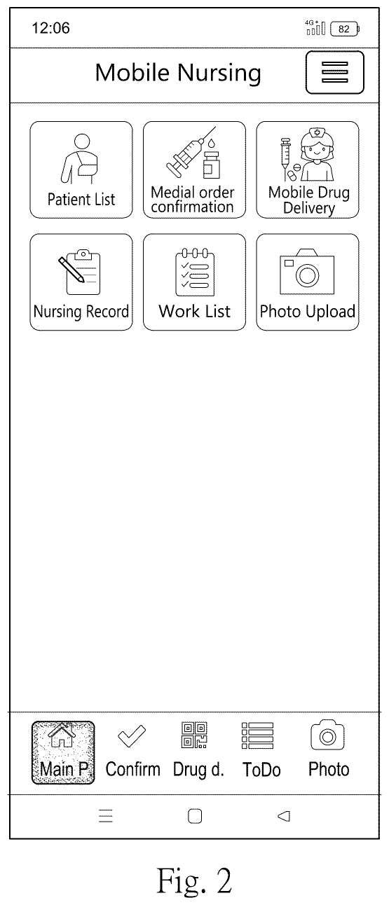

Please refer to FIG. 1 and FIG. 2. For the system, the nursing staff creates a medical care information system, using the mobile device 10 and the user interface module 30 to input user information electrically connected to the mobile device, and operate the nursing staff through the hospital management. Extract data from the hospital information system 20 database to operate six modules including patient list module 31, medical order confirmation module 32, nursing record module 33, mobile drug delivery module 34, work list module 35, upload photo module 36, the following coordination The tab diagrams of each module illustrate how to provide nursing staff with accurate and effective care for patients.

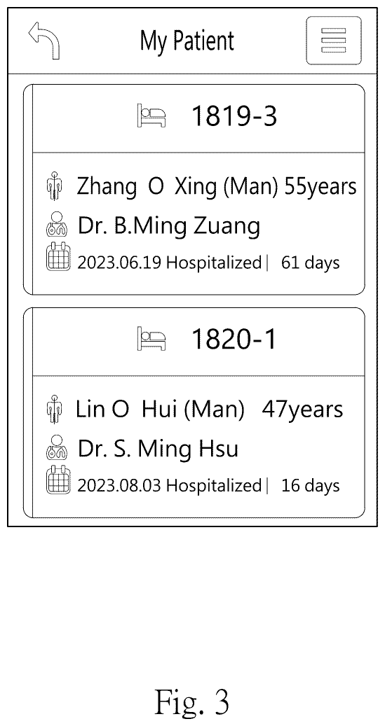

Please refer to FIG. 3. The screen shows the patient list module 31 displays “My Patients”, and presents a list of all patients cared for by the person who operates the mobile device 10, that is, the nursing staff, and visually presents it with graphics and colors; The first layer of the patient list contains important information such as the patient's name, gender, age, attending physician, admission date, and the number of days he has been hospitalized. The name is presented in accordance with the hospital's patient privacy protection policy; the second layer of the patient list contains TPR, Functional items such as medication records, nursing records, medical records, test reports, examination reports, operation records, consultation records, pathology reports, radiology reports, EKG, VS comment, etc. Each function item can be clicked to read or inquire; through each the records of the nursing team and the patient list have the function of team communication. After the nursing staff administers the medicine, the doctor can learn from the medication record; after the patient's wound is photographed and uploaded, the doctor, specialist nurse, and nursing staff can read and learn about the patient's condition.

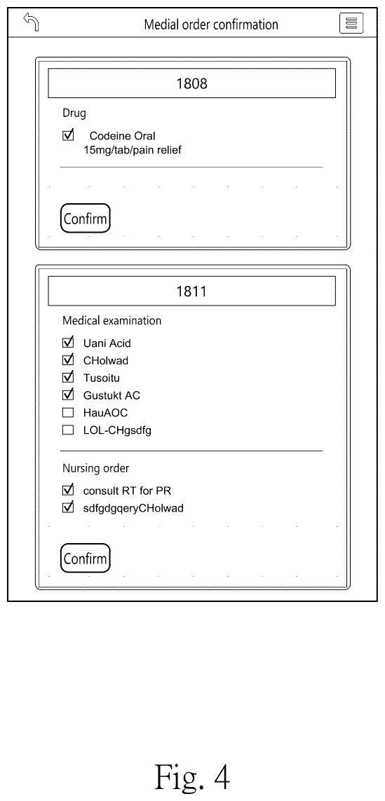

Please refer to FIG. 4. The screen shows the medical order confirmation module 32 shows “Medical order confirmation”. When the doctor issues the medical order, the medical order confirmation module 32 will push and sound notification to the specific nursing staff to remind them to click and confirm. Physicians no longer need to spend time looking for the primary nursing staff; nursing staff can take care of patients according to their needs, and click to confirm single or multiple medical orders for a single patient, or all medical orders for a single patient, or all medical orders for all patients. Confirmation is fast and instant. Nursing staff no longer need to look for a desktop computer to confirm the medical order; after the medical order is confirmed, it is linked to the hospital information system 20 database to complete the update simultaneously.

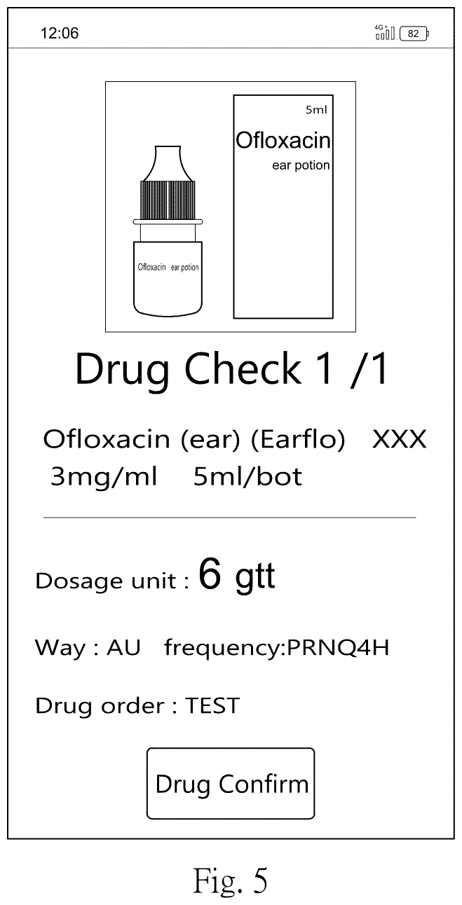

Please refer to FIG. 5. The screen shows the mobile drug delivery module 33 shows “Mobile Drug Delivery”. The mobile drug delivery module 33 has the function of summarizing all the patient's medication orders and displays the time points that should be administered according to the frequency of administration. The status of the dosing time point is visually displayed in color, and if the doctor has prescribed medication instructions, it will also be displayed; all drugs have pictures for verification. Click on the drug picture to connect to the hospital drug query system, and you can query the detailed information of the drug. or drug instructions to provide more complete dosing information; the nurse in charge clicks on the patient who wants to administer medication and opens the mobile medication screen. After opening the camera through the mobile device 10 to scan the medicine bag/medicine bag barcode, the mobile medication module 33 actively checks the correctness of the medicine. The picture of the drug and delivery details are displayed on the screen for the nursing staff to check. If the drug is wrong or the time is wrong, it will be intercepted. After checking, the nurse in charge must click “Drug is correct” to continue to the next step; if the nursing staff If the medication is interrupted and the medication procedure is not completed, the nursing staff can click “Leave” to re-execute the check to improve medication safety; the nurse in charge checks the medicine and brings it to the patient's bedside to perform patient identification and turns on the camera through the mobile device 10 to scan the patient's hand band barcode. The mobile medication module 33 actively checks the patient's accuracy and intercepts if the patient makes a mistake. If it is correct, the responsible nursing staff will perform the medication and click=Delivery Complete”. The mobile drug delivery module 33 breaks through the space limitation, making medication safe and easy; after the mobile medication is completed, it will be linked to the hospital management system 20 database for synchronization The update is completed and the “Medication Record” in the patient list module 31 is linked for review to increase team communication.

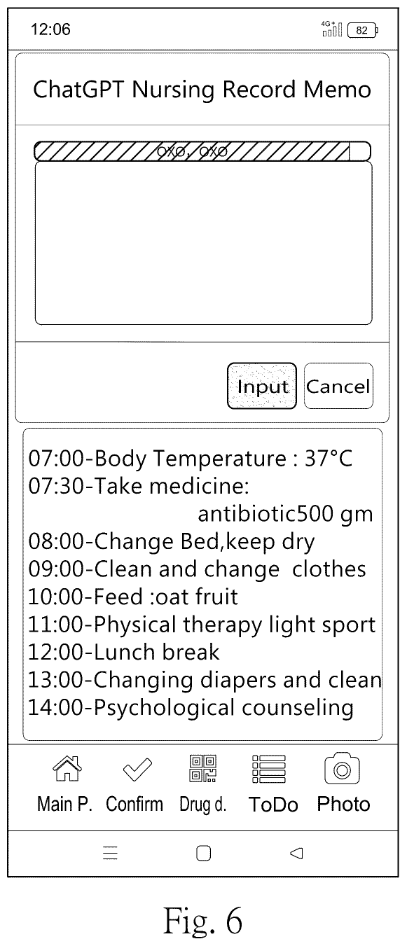

Please refer to FIG. 6. “Nursing Record” is displayed on the screen shows the nursing record module 34. The nursing record module 34 is a time axis with a single patient, providing the primary nursing staff or assisting nursing staff during their shift. The patient's event record can be input in real-time by voice-to-text or manually. The event record has a modification/save function; the responsible nursing staff can use the built-in ChatGPT organizing function in the event record to check the event records recorded in real time in the timeline and save them. The nursing records that are organized into focus records and brought into the hospital management system 20 database are not only synchronized with the hospital management system 20 database, but also synchronized with the “nursing record” in the patient list module 31 for the relevant care team to review, achieving For accurate and real-time purposes; ChatGPT organizes the event records selected by the responsible nursing staff into the DART record format of the nursing record specification according to specific instructions, and brings them into the hospital management system 20 nursing record database according to the format to complete the update simultaneously.

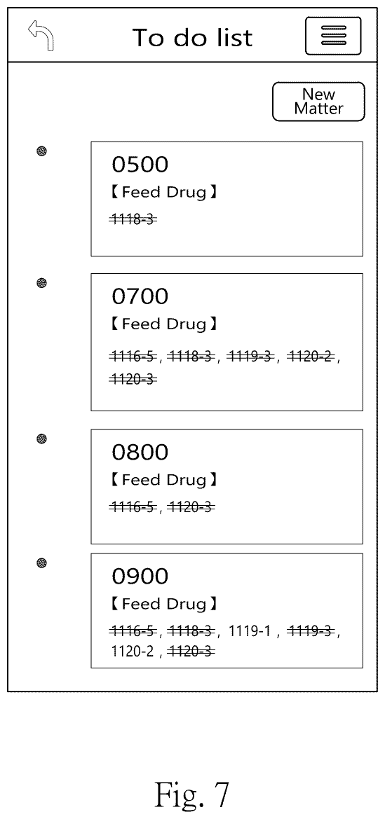

Please refer to FIG. 7. The “work list” is displayed on the screen shows the work list module 35. The work list module 35 is an exclusive personal assistant centered on a specific nursing staff to remind the responsible nursing staff of the treatment. It is used to do things; the work list is centered on the work time axis of the main nursing staff, and the work items that should be executed are automatically imported in conjunction with the doctor's orders, and arranged into the work time axis of the main nursing staff according to the scheduled execution time, with the bed number and to-do projects are presented concisely; according to the scheduled execution time, from half an hour before the project to one hour after the project, a yellow background is displayed on the timeline, and projects outside this time period are displayed with a white background to remind the responsible nursing staff of the currently scheduled processing projects. The items that have been executed are shown in red letters and drawn with a red line to remind the responsible nursing staff of the bed number and items that have not yet been executed; if only one of all the drugs for the same patient at that time has not been completed, it will not be completed. It turns into red letters and draws a red line to remind the nursing staff in charge to pay attention to whether there are any missed items; the work list also provides a work schedule for non-medical orders. The nursing staff in charge can plan care according to the matters that need to be paid attention to in the previous shift. For the care that the patient must perform, etc., arrange the scheduled execution items into the timeline of the work list, and manually click to complete them after execution.

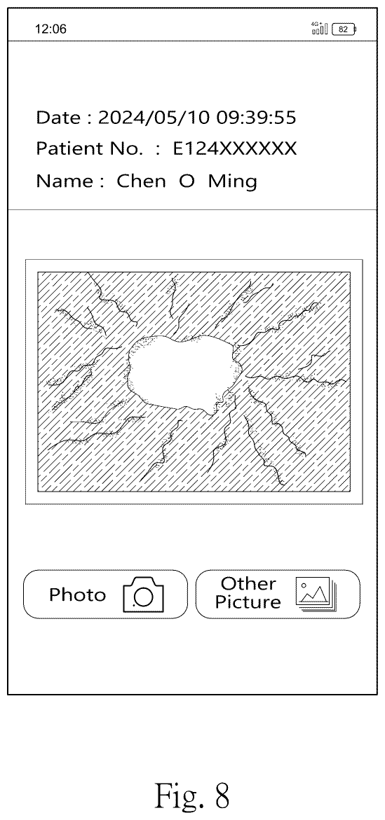

Please refer to FIG. 8. The screen shows the upload photo module 36 shows “upload photos”. The upload photo module 36 provides the functions of taking pictures of patient wounds and recording images of special events; click “Upload Photos” to scan the barcode on any patient's handband with the mobile device 10 to instantly query the photos that the patient has uploaded; scan the barcode on any patient's handband with the mobile device 10 and click “Photo Upload” to automatically turn on the camera function of the mobile device 10, click “OK” after taking pictures to automatically upload the photos; the uploaded photos are automatically synchronized to the database of the hospital management system 20 for query or saved to a specific medical record location, and are automatically imported into the “Patient List” of the patient list module 31 “Medical records” inside are available for review to increase team communication.

The above-mentioned mobile device 10 may be a smart phone or a pad computer, and any device with an Internet of Things function developed in future technologies may be applicable.

What is claimed is:

1. A nursing care information system including:
a mobile device, including:
a user interface module electrically connected to the mobile device for inputting user information;
a patient list module electrically connected to the user interface module to provide a list of patients cared for by nursing staff;
a medical order confirmation module electrically connected to the user interface module and used to push notifications and sound notifications to nursing staff, as well as reminders and confirmations;
a nursing record module electrically connected to the user interface module and used to provide nursing staff with input of patient event records;
a mobile drug delivery module electrically connected to the user interface module and used to provide medical advice to patients under the care of nursing staff;
a work list module electrically connected to the user interface module and used to provide and remind caregivers of to-do items; and
a photo module electrically connected to the user interface module and used to provide nursing staff with the ability to take photos of patient wounds and image records of special events.

2. The nursing care information system as claimed in claim 1, wherein the first-level tab of the patient list module displays the patient's name, gender, age, attending physician, admission date, and the number of days he or she has been hospitalized.

3. The nursing care information system as claimed in claim 1, wherein the second-level tab of the patient list module displays TPR, medication records, nursing records, medical records, test reports, examination reports, operation records, consultation records, pathology reports, radiology reports, EKG, VS comment and other functional items, each all function items can be clicked to browse or inquire.

4. The nursing care information system as claimed in claim 1, wherein the medical order confirmation module will push and sound notifications to specific nursing staff after the doctor writes the medical order to remind them to click and confirm.

5. The nursing care information system as claimed in claim 1, wherein the mobile drug delivery module displays the time points that should be administered according to the frequency of administration, the status of the administration time points is visually presented in color, if the doctor has prescribed medication instructions, it will also be displayed and pictures of the drugs are provided for verification, click on the drug picture to connect to the hospital's drug query system, where you can query the drug's detailed information or drug instructions, providing more complete dosing information.

6. The nursing care information system as claimed in claim 1, wherein the mobile drug delivery module has a mobile medication screen tab, after opening the camera on the mobile device to scan the barcode of the medication bag/medication bag, it will automatically check the accuracy of the medication, at the same time, the screen will display the picture of the medication and detailed medication information, for caregivers to check.

7. The nursing care information system as claimed in claim 1, wherein the mobile drug delivery module is used by nursing staff to check the medicine and then bring it to the patient's bedside to perform patient identification and check the patient's accuracy, if the patient makes a mistake, it will be intercepted.

8. The nursing care information system as claimed in claim 1, wherein the nursing record module allows nursing staff to instantly enter patient event records via voice-to-text or manual input.

9. The nursing care information system as claimed in claim 1, wherein the work list module is used to remind the responsible nursing staff of to-do items.

10. The nursing care information system as claimed in claim 1, wherein the photo upload module provides patient wound photos and image records of special events.
